SEQUENCE LISTING (4 sequences)

SEQ ID NO: 1 - DNA, 8 bp, Artificial sequence
  Synthetic polynucleotide
atttttat

SEQ ID NO: 2 - DNA, 8 bp, Artificial sequence
  Synthetic polynucleotide
gtttttgt

SEQ ID NO: 3 - DNA, 16 bp, Artificial sequence
  Synthetic polynucleotide
atttttatgtttttgt

SEQ ID NO: 4 - DNA, 16 bp, Artificial sequence
  Synthetic polynucleotide
atttttatatttttat

(19) United States
(12) Patent Application Publication
(10) Pub. No.: US 2008/0261204 A1
(43) Pub. Date: Oct. 23, 2008

(54) Polynucleotide Ligation Reactions

(75) Inventor: Preben Lexow, Oslo (NO)

Correspondence Address: MARSHALL, GERSTEIN & BORUN LLP, 233 S. WACKER DRIVE, SUITE 6300, SEARS TOWER, CHICAGO, IL, 60606, US

(73) Assignee: LINGVITAE AS, Oslo (NO)

(21) Appl. No.: 10/586,556

(22) PCT Filed: Jan. 21, 2005
(86) PCT No.: PCT/GB05/00218

§ 371 Date: Jan. 7, 2008

Related U.S. Application Data
(60) Provisional application No. 60/560,321, filed on Apr. 6, 2004.

(30) Foreign Application Priority Data
Jan. 23, 2004 (GB) 0401524.4

(51) Int. Cl. C12Q 1/68; G01N 33/50

(52) U.S. Cl. 435/6; 435/7.1; 436/501

(57) ABSTRACT

The method of the invention is useful in quantifying the absolute or relative number of unique molecules present in a sample after carrying out an analysis procedure on the sample, and comprises the steps of: (i) attaching a unique molecular tag to substantially all of the molecules in the sample; (ii) carrying out the analysis procedure using the molecules of the sample; and (iii) on the basis of the molecular tags determining the absolute or relative number of unique molecules present in the original sample which underwent the analysis procedure.

### FIELD OF THE INVENTION

This invention relates to a method for quantifying the absolute and/or relative numbers of molecules that undergo an analysis procedure; and allows the tracking of an individual molecule during an analysis procedure. The invention is useful especially in the analysis of polynucleotides and proteins.

### BACKGROUND TO THE INVENTION

Methods for molecular analysis often require that the original target molecules must be subject to various processes such as amplification and labelling before the analysis itself can take place. It is, however, a problem that the efficiency of such processes are subject to variation. For example, in an amplification process one target molecule in a sample may be copied more times than another target molecule, thereby making it difficult to measure the absolute and relative amounts of the different target molecules that were present in the original sample. Furthermore, the analysis procedure itself often results in the mixing of molecules such that it is not possible to maintain information on each individual molecule. Previously disclosed methods for tagging molecules have not addressed this problem.

Examples of methods of tracking and identifying classes or sub-populations of molecules using oligonucleotide tags have been disclosed in U.S. Pat. No. 5,604,097 and U.S. Pat. No. 5,654,413. U.S. Pat. No. 5,604,097 and U.S. Pat. No. 5,654,413 disclose methods for sorting sub-populations of identical polynucleotides from a sample onto particular solid phase supports. This is achieved by attaching an oligonucleotide tag from a repertoire of tags to each molecule in a population of molecules so that substantially all of the same molecules or same sub-population of molecules have the same tag attached, and substantially all different molecules or different sub-populations of molecules have different oligonucleotide tags attached. Furthermore, each oligonucleotide tag from the repertoire comprises a plurality of sub-units and each sub-unit consists of an oligonucleotide having a length from 3 to 6 nucleotides or from 3 to 6 base pairs; the sub-units being selected to prevent cross-hybridisation. The molecules or sub-populations of molecules may then be sorted by hybridising the oligonucleotide tags with their respective complements found on the surface of a solid support.

The methods allow tracking and sorting of classes or sub-populations. However, there is no disclosure of sequencing the tag on each molecule so that individual molecules can be identified.

### SUMMARY OF THE INVENTION

The present invention is based on the realisation that the absolute and/or relative amounts of a unique target molecule can be determined and that individual molecules within a population can be tracked throughout an analysis procedure, by using a molecular tag that is unique to each specific molecule.

According to a first aspect of the invention, a method of quantifying the absolute or relative number of unique molecules present in a sample after carrying out an analysis procedure on the sample, comprises the steps of:

(i) attaching a unique molecular tag to substantially all of the molecules in the sample;

(ii) carrying out the analysis procedure using the molecules of the sample; and

(iii) on the basis of the molecular tags determining the absolute or relative number of unique molecules present in the original sample which underwent the analysis procedure.

The ability to determine the amounts of a unique molecule present in an original sample after amplification is of benefit in many processes. For example, it can be used for transcription analysis in order to measure the amounts of different mRNA classes.

According to a second aspect of the present invention, a method for determining the sequence of a polynucleotide in a sample, comprises the steps of:

i) attaching a unique molecular tag to substantially all the polynucleotides in the sample;

ii) fragmenting the amplified polynucleotides; and

iii) sequencing at least those fragmented polynucleotides that comprise a molecular tag, wherein, on the basis of the molecular tags, the sequence information for each individual polynucleotide can be collated, for example using a computer programme.

This is useful in simplifying the reconstruction of sequence data from individual sequence fragments, particularly in de novo sequencing.

According to a third aspect of the present invention, a method for detecting the presence of a protein in a sample, comprises contacting the sample with two or more protein binding molecules each having affinity for different parts of the target protein, wherein the protein-binding molecules comprise a polynucleotide molecular tag and wherein, on binding of at least two protein-binding molecules to the target protein, the molecular tags can be ligated in a subsequent ligation step, and the ligated polynucleotide detected, characterised in that the ligated polynucleotide comprises a sequence that identifies the class of target protein and the individual protein.

According to a fourth aspect of the present invention, a method for detecting the presence of specific proteins present on the outer-surface of a cell, comprises:

(i) contacting the cell with a sample comprising different protein-binding molecules, each protein-binding molecule comprising a polynucleotide molecular tag of defined sequence;

(ii) carrying out a ligation reaction to ligate adjacent polynucleotides; and

(iii) detecting the ligated polynucleotide(s) and determining the presence of the outer-surface proteins;

wherein the polynucleotide molecular tags comprise a nucleotide sequence that identifies the class of outer-surface protein and the individual protein.

### DESCRIPTION OF THE DRAWINGS

The invention is described with reference to the accompanying drawings, wherein:

FIG. 1 illustrates how the molecular tags are used to identify both the class of molecule and the individual molecule;

FIG. 2 illustrates how a further part of the molecular tag can be used to provide sequence information for each molecule; and

FIG. 3 illustrates how molecules that are attached to substrates such as beads, microbes or cells can be quantified; and

FIG. 4 illustrates how the molecular tags can be used to identify outer-surface proteins, using a ligation reaction.

### DETAILED DESCRIPTION OF THE INVENTION

The present invention is used in the analysis of unique molecules. The molecule may be any molecule present in a sample which undergoes an analysis procedure. In a preferred embodiment, the molecules are polymers. The terms “polymer molecules” and “polymers” are used herein to refer to biological molecules made up of a plurality of monomer units. Preferred polymers include proteins (including peptides) and nucleic acid molecules, e.g. DNA, RNA and synthetic analogues thereof, including PNA. The most preferred polymers are polynucleotides.

The term “molecular tag” is used herein to refer to a molecule (or series of molecules) that imparts information about a target molecule to which it is attached. The tag has a unique defined structure or activity that represents the attached individual target molecule. The tag may also contain a second defined structure that represents the class (or sub-population) of target molecule. If the sample comprises a single class of molecules, this additional structure is not required and the tag may comprise only the unique portion.

A sample identification portion may also be used to retain information on the origin of the target molecule. In this way, it will be possible to retain the possibility of tracking back, after several assays or procedures using the target molecule, to identify the original sample from which the target molecule was taken. For example, the sample identification portion may be specific for an individual patient from whom a biological sample is taken. Accordingly, assays may be performed at the same time on samples from numerous patients, and the results analysed with the knowledge of where each target molecule was obtained. This is beneficial also in preventing erroneous analyses of a mis-labelled sample.

The molecular tag is stated to be attached to “substantially” all of the molecules in the sample. It is preferred if the tags are attached to greater than 80% of the molecules in the sample, more preferably 90%, 95% or 98% and most preferably at least 99% of the molecules. In the eventual read-out step, the tags on the molecules will be determined. It is preferred that at least 80% of the tags in the final sample are determined, preferably at least 90% and most preferably at least 95%. It is desirable to carry out the read-out step in a way that ensures that each tag in the original sample is read at least once. This ensures that each tag is identified at least once. A statistical analysis can then be made.

The molecular tag may be any biological molecule that can impart the necessary information about the target molecule. Preferably, the molecular tag is a polymer molecule that can be designed to have a specific sequence which can therefore be used in the identification of the attached molecule. In the most preferred embodiment, the molecular tag is a polynucleotide that comprises a nucleic acid sequence that is unique and specific for the individual target to which the molecular tag is attached. This tag may also comprise a further nucleic acid sequence which represents the class (or sub-population) of sample molecules and also, optionally, a sample identification portion. The polynucleotide may be of any suitable sequence. Any suitable size of polynucleotide may be used. The size will depend in part on the number of different target polymers to be “tagged” as a unique sequence is required for each (or substantially each) target.

In the context of polynucleotide tags, these can be amplified, eg by means of a polymerase reaction, so that the tags can be determined in a later read-out step. On read-out, the tags do not therefore need to be attached to the target molecule. In this embodiment, it may be necessary to add to the tag a sequence that binds to an appropriate primer for use in the polymerase reaction. This sequence may be present on the tag prior to addition to the target, or may be added (eg via ligation) once the tag has been bound to the target.

In a further embodiment, the molecular tag is or comprises an aptamer with affinity for the sample molecule. In a preferred embodiment, the molecular tag comprises a target-specific aptamer, (which specifically binds the target molecule) and a unique polynucleotide tag. Aptamers known to recognise biomolecules and methods of their production are well known in the art, for example in WO-A-00171755, the content of which is hereby incorporated by reference.

Alternatively, the tag may be or may comprise a protein. Preferably, the tag in this case is or comprises an antibody which has affinity for the sample molecule.

It is envisaged that a tag could be formed by combining any of the above into a single moiety, for example an antibody linked to a polynucleotide or an aptamer linked to a polynucleotide.

Preferably, there is a large excess of unique tags with respect to the sample molecules, such that when attachment occurs it is statistically likely that substantially all sample molecules will be attached to a different, unique tag.

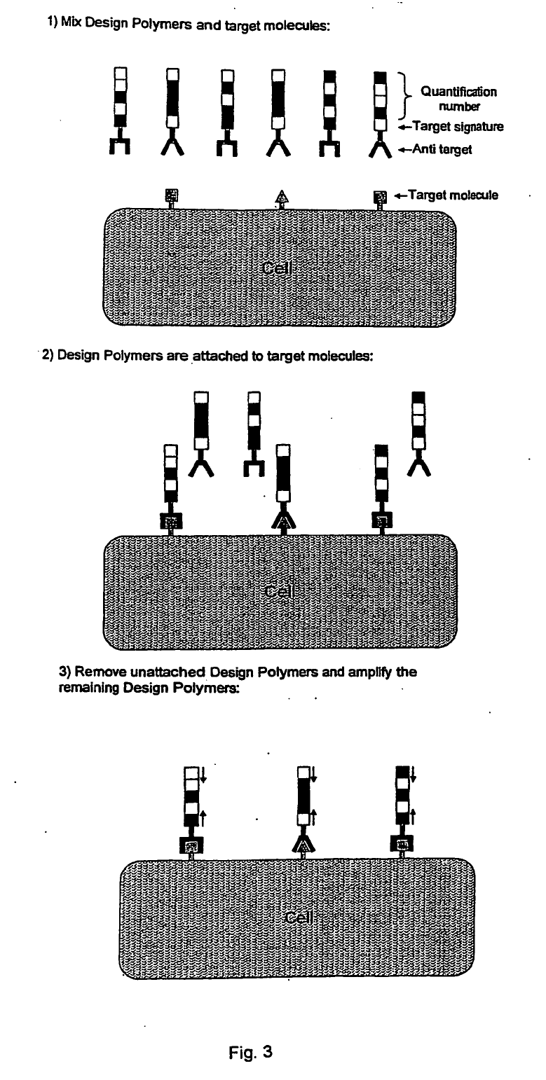

The sample may comprise molecules that are all identical or substantially similar, or molecules from different populations, i.e. there may be a single class or several classes of molecule in the sample. Molecules in the same class are identical or have a common attribute, for example a population of identical DNA molecules amplified by PCR, or a mixed population of mRNA transcripts which, although comprising different sequences, all have the common attributes of mRNA and therefore belong to the same class. Molecules of different classes differ in structure or some other attribute, for example a cell surface (as depicted in FIG. 3) contains proteins, carbohydrates, glycoprotein, lipids and other biological molecules which all have distinct structures and attributes. These may be determined using the methods of the invention. Further examples of a sample containing different classes of molecules may be DNA/RNA mixtures, cell lysates, or samples containing different classes of proteins.

It will be apparent to one skilled in the art whether the sample comprises a single class or multiple classes of molecule.

The method of the invention is to be used to “tag” target molecules in a sample prior to analysing the target molecules.

Tagging may be carried out by any suitable method, including chemical or enzymic methods, for linking the molecular tag with the target molecule. In the context of a nucleic acid target polymer and a polynucleotide tag, the tagging process may be carried out by suitable ligase enzymes. The tag will usually be ligated onto one of the terminal ends of the target. For example, double stranded polynucleotides may be treated to create single stranded overhangs, which may hybridise with complementary overhangs on the polynucleotide tags and be ligated using a suitable ligase enzyme. Any method of generating the single stranded overhangs may be used, a preferred method is the use of class IIS restriction enzymes.

In the context of aptamers or antibodies, the tag is attached to the sample molecule by means of the specific target-aptamer/antibody interaction.

The molecular tag may also be attached to a different molecule, which is used to bind to the target molecule. For example, the tag may be a polynucleotide attached to protein-binding molecule (e.g. antibody), which has affinity for a particular target.

The molecular tag may be in a form that represents a binary system, wherein each tag is represented by a series of “0”s and “1”s, allowing a large amount of data to be contained within a small number of tag components. For example, different combinations of “0” and “1” may be formed to provide unique sequences of “0” and “1” that can be used as unique tags.

Preferably, the signals “0” and “1,” are represented by different oligonucleotide sequences, for example:

| | |
|---|---|
| “0” = | ATTTTTAT |
| “1” = | GTTTTTGT |
| ATTTTTATGTTTTTGT = | “0, 1” |
| ATTTTTATATTTTTAT = | “0, 0” |

The molecular tag is, or may comprise, repeating units of nucleotide sequence, with the combination of units forming a unique sequence that can be characterised to identify, for example, the class of target molecule associated with the molecular tag, the individual target molecule, and if desirable, the sample from which the target was taken.

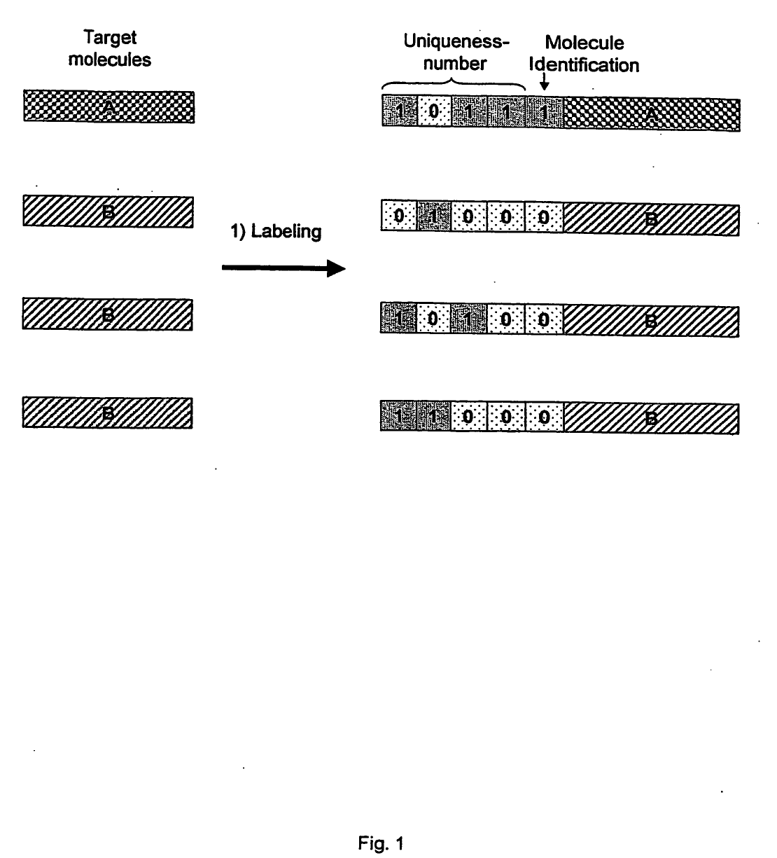

This system is advantageous since many unique tags can be created using only two units. This is illustrated by FIG. 1.

When the tag comprises a unique series of “0”s and “1”s according to this binary system, the unique portion of the tag is referred to herein as the “uniqueness number portion”. According to the binary system, a preferred tag may comprise a uniqueness number portion, which identifies the individual molecule, and if the sample comprises several classes of molecule, a second defined binary sequence may represent the “molecular class portion”, defining each class of sample molecule. Each class of sample molecule is therefore tagged with a different molecular class portion, and each sample molecule within the class has a different uniqueness number portion. This is illustrated by FIG. 1.

Attaching the unique portion (“uniqueness number portion” if the binary system is used) of the molecular tag to the sample molecule occurs prior to any analysis procedure. The sample identification portion may be attached to the sample molecule at any point before, during or after the analysis procedure.

The analysis procedure may be any procedure used to analyse the molecules.

When the sample molecules are biological molecules such as proteins and polynucleotides, there are a great number of analysis procedures present in the art that would benefit from having each sample molecule individually tagged. Methods of characterising the physical, chemical and functional properties of a molecule are within the scope of “analysis procedures”. Such techniques are well known to those in the art. Sequencing of biological polymers may be such an analysis procedure.

In one embodiment, the molecular tags are polynucleotides and may be used in a proximity ligation reaction, for example as disclosed in Gullberg et al, PNAS, 2004; 101(22): 8420-8424, and WO-A-01/61037, the content of each being incorporated herein by reference. In this embodiment, a target protein is contacted with two or more protein-binding molecules each comprising a polynucleotide molecule. On binding to the target molecule, the polynucleotides are brought into proximity and can subsequently be ligated using conventional ligation procedures. The ligated polynucleotides can then be identified, on the basis of the nucleotide sequence; for example the polynucleotide can be amplified in a polymerase reaction and the absolute or relative number of polynucleotides can be determined on sequencing. The polynucleotides will be designed to incorporate sequences that provide information on the class of target molecule, the individual molecule and, if necessary, the sample from which the target molecule was obtained. The polynucleotides may therefore be in the “binary” form as disclosed herein. The protein-binding molecules may be, for example, antibodies or aptamers that bind to different epitopes on the target protein.

The analysis procedure may also comprise the separation of a mixture of molecules, the division of molecules into discrete populations or the amplification of molecules, in particular polynucleotides. These analysis procedures may be applied in many techniques, for example quantifying polynucleotides using the method of the present invention can be used in transcription analysis of cDNA or mRNA, to determine the number of transcripts. Microbial floras may be analysed in a similar fashion; based upon analysis of genomic DNA from different microbial species it is possible to generate unique transcript profiles for each species that can be verified using tags as described by the method of this invention. Quantifying polynucleotides may also be used in ribosomal analysis based on rRNA tagging and detection.

Quantifying molecules that cannot themselves be amplified (as illustrated in FIG. 3) may be applied in the analysis of membrane-bound ligands such as proteins, carbohydrates and lipids, and may also be applied in the analysis of biological molecules cross-linked to a surface.

In a preferred embodiment, the analysis procedure comprises amplification by Polymerase Chain Reaction (PCR). Depending on the nature of the molecular tag, only the tag itself or the tag and sample molecule may be amplified.

For example, if the tag comprises an antibody attached to a unique polynucleotide, wherein the antibody recognises and binds a protein, amplification by PCR will amplify the unique polynucleotide only. In this embodiment, after contacting the tag to the sample molecule, non-bound tags are removed from the reaction mix. Suitable methods of removal will be apparent to the skilled person. Amplification by PCR is then carried out, wherein only the polynucleotide tag is amplified. The information contained within the tag(s) after amplification is sufficient to determine the number of different molecules present in the original sample.

Alternatively, if both the target molecule and tag are polynucleotides, PCR will result in amplification of both the tag and attached sample molecule. Non-bound tags may again be removed before amplification. In this embodiment, the sample molecules are amplified and may be further analysed or used, whilst the tags (which have also been amplified) contain the information on the number of different molecules present in the original sample.

The method of the invention may also be used to identify multiple outer-surface proteins (or other molecules) present on a cell. In this embodiment, the molecular tag is, or is attached to, a protein-binding molecule which can be brought into contact with the cell. Those tags that are bound to outer-surface proteins can be identified in a later identification step. For example, if the tag is a polynucleotide, this can be amplified in a subsequent polymerase reaction.

In a further development of this procedure, multiple outer surface molecules can be identified in one assay by ligating the polynucleotide tags bound to outer surface molecules. This is carried out as follows:

(i) contacting the cell or membrane with a sample comprising different molecule-targeting moieties, each moiety comprising a polynucleotide molecular tag of defined sequence;

(ii) carrying out a ligation reaction to ligate adjacent polynucleotides; and

(iii) detecting the ligated polynucleotide(s) and determining the presence of the outer-surface or membrane molecules;

wherein the polynucleotide molecular tags comprise a nucleotide sequence that identifies the class of outer-surface molecule and the individual molecule.

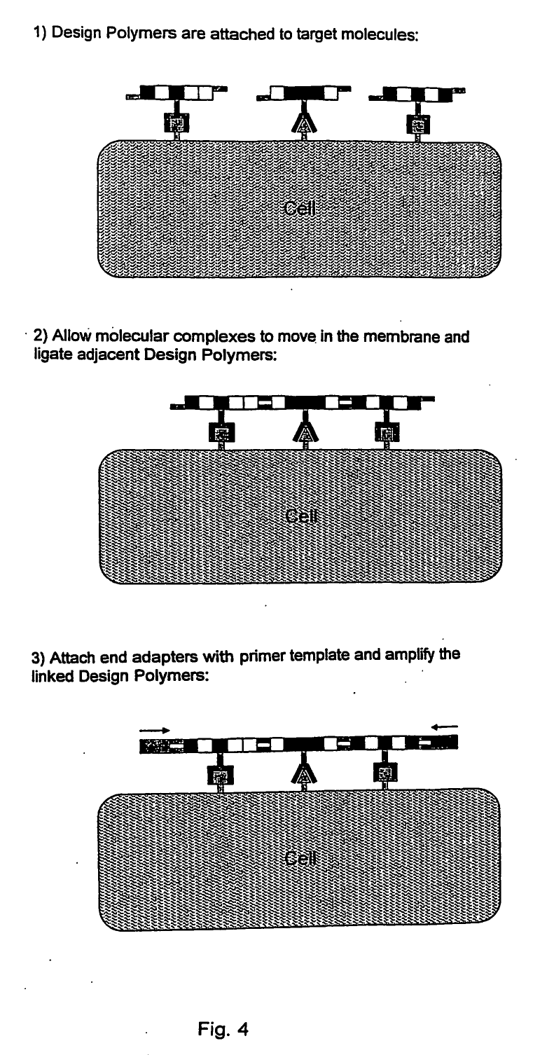

The reference to “adjacent” is not intended to imply that the outer-surface molecules are located immediately next to each other. Rather, the term is intended to mean that ligation can take place if the polynucleotide tags can be placed proximal to each other, to allow ligation to occur. This concept is illustrated in FIG. 4.

In a further preferred embodiment, the analysis procedure comprises detection of the tagged-molecule using a nano-pore detection system. This technique is used when information on each tagged molecule is required. Nanopore methods of detection are well known in the art, and are described in Trends Biotechnol. 2000 April; 18(4):147-51, the content of which is incorporated herein by reference.

Suitable nanopores for polynucleotide detection include a protein channel within a lipid bilayer or a “hole” in a thin solid state membrane. Preferably the nanopore has a diameter not much greater than that of a polynucleotide, for example in the range of a few nanometres. As the tagged polynucleotide enters a nanopore in an insulating membrane, the electrical properties of the pore alter. These alterations are measured and as the tagged polynucleotide passes through the pore, a signal is generated for each nucleotide.

The method of the present invention allows an entire sample of polymers to undergo nanopore analysis without losing information on the origin of each molecule, and whilst still being able to determine the number of different molecules present in the original sample, after nanopore analysis.

Once the analysis procedure has been carried out, the molecular tags are determined. The method of determination will differ depending on the tag used. When the tag is a polynucleotide, it can be characterised by sequencing. Methods of sequencing are well known to those skilled in the art and suitable techniques will be apparent.

Once the sample has been tagged, it is possible to repeat the method, if required, and then the resulting product analysed by determining the molecular tag(s).

The method may be carried out in solution or where the sample molecules are attached to a surface. Such surfaces include biological membranes, beads or living cells. For example, the number of different proteins on a cell surface may be detected, by attaching a unique tag to each class of proteins, amplifying and detecting the number of different unique tags. When the sample molecule is attached to a surface, the molecular tag may comprise an antibody as shown in FIG. 3, although other molecular tags such as aptamers and polynucleotides may also be used. In a preferred embodiment the sample molecule is not attached to a support surface at the stage of the read-out analysis. The sample molecules may therefore be contained in a heterogeneous population with other different sample molecules. The tags of individual molecules can be determined (read) and the information collected on computer to track the molecule and its characteristics.

FIG. 3 illustrates a method for quantifying target molecules that are attached to a substrate such as beads, microbes or cells. The method may be used to quantify molecules such as proteins bound to a cell membrane as follows:

i) The cell is mixed with molecular tags each of which comprises a moiety (antibody or aptarmer) with the ability to bind to a specific target molecule, a unique polynucleotide representing the specific target molecule and a sample identification portion. In order to reach saturation of bound target there is a large surplus of molecular tags versus target molecules.

ii) Any unattached molecular tags are removed from the reaction mix after the binding reaction has reached saturation.

iii) The polynucleotide part of the molecular tag is amplified and analysed. The number of unique molecular tags that can be associated with a specific target label gives the original number of target molecules.

When the sample molecule is in solution, for example when measuring the number of different mRNA classes in an analysis of transcription, the molecular tag may comprise an aptamer and/or a polynucleotide although other molecular tags such as antibodies may also be used.

1. Target molecules and molecular tags are mixed.
A solution containing the target molecules (e.g. macromolecules such as proteins) is mixed with a large surplus of molecular tags comprising a moiety (e.g. an aptamer) that has the ability to bind to the target molecules with specificity and which comprises a unique polynucleotide portion.

2. Molecular tags are allowed to bind target molecules.

3. Unbound molecular tags are removed.
This can be achieved, for example, using gel electrophoresis, spin columns or other separation methods known in the art.

4. Molecular tags bound to target molecules are amplified and the number of unique tags is determined.
The unique tags may, then be amplified by PCR before a representative number of the amplified molecular tags are further analysed.

When the sample molecules are polynucleotides, it is possible to use more than one polynucleotide tag in order to increase the specificity of the tagging reaction. Two different tags, each comprising sequences complementary to different but adjacent sequences on the sample polynucleotide and each comprising unique tag sequences, may be hybridised to the sample polynucleotide. These two tags are then ligated together and amplified, as a single polynucleotide, by PCR. The ligation step increases the specificity of the quantification, as two specific tags are required to hybridise compared to the single tag normally used. Only correctly hybridised, adjacent tags will be ligated and amplified.

1. Sample polynucleotides and polynucleotide tags are mixed:
Single stranded sample polynucleotides are contacted with two polynucleotide tags each comprising a sequence that can hybridize with specific adjacent parts of the sample sequence. Successful hybridization of the two different polynucleotide tags will bring them into contact with each other, allowing ligation to take place.

2. Polynucleotide tags are hybridised to sample polynucleotides and ligated:
Only the hybridised and ligated polynucleotide tags can be amplified by PCR. The ligation step increases the specificity of the quantification procedure.

3. Polynucleotide tags bound to sample polynucleotides are amplified and the number of unique tags determined.

FIG. 1 illustrates a method of the first aspect of this invention wherein the analysis procedure is amplification. The first, pre-amplification sample contains four target polymer molecules, one “A” DNA molecule and three “B” DNA molecules. Prior to the amplification reaction a molecular tag is incorporated onto each target polymer molecule. The molecular tag comprises two portions. One portion is the sample identification portion which identifies the target polymer type. In this example the molecular tag uses a binary system and subunit “1” represents polymer type “A”. Molecular tag subunit “0” represents target polymer type “B”. Another portion of the molecular tag, the “uniqueness number portion”, identifies the individual target polymer. As can be seen in FIG. 1 each of the “B” target DNA molecules has a molecular tag containing a different uniqueness number portion. The molecular tags are incorporated on the targets by ligation.

Once each target polymer molecule has been tagged, the tags and attached targets are amplified using the polymerase reaction. The amplification reaction is random and in any given sample one target polymer molecule may not be copied exactly the same number of times as other target polymer molecules.

After amplification, if a given number of the amplified molecular tags are read, ensuring that each unique molecular tag is read at least once with a high statistical probability, it is possible to deduce the absolute and/or relative amount of “A” and “B” molecules by counting how many unique tags are associated with molecules “A” and “B” respectively.

In this way information is gained about the composition of the first, pre-amplification sample and about the amplification step itself.

A further embodiment of the invention comprises a method of tracking the presence and origin of an individual molecule and/or copies and/or fragments thereof. The sample molecules may be polymeric nucleic acids, which are tagged with oligonucleotide molecular tags as previously described. A preferred analysis procedure is amplification of the tag and attached sample molecule, followed by fragmentation of the amplified polymers; for example as used in ‘de novo’ sequencing methods. The result of this fragmentation is a selection of labelled polynucleotides of different lengths, with all molecules from the same origin (parent molecule) containing the same label, allowing the origin of each molecule to be traced.

The amplified products may be modified in further processes, and the modifications monitored by the incorporation of additional tags. For example, portions of each amplified product may be sequenced.

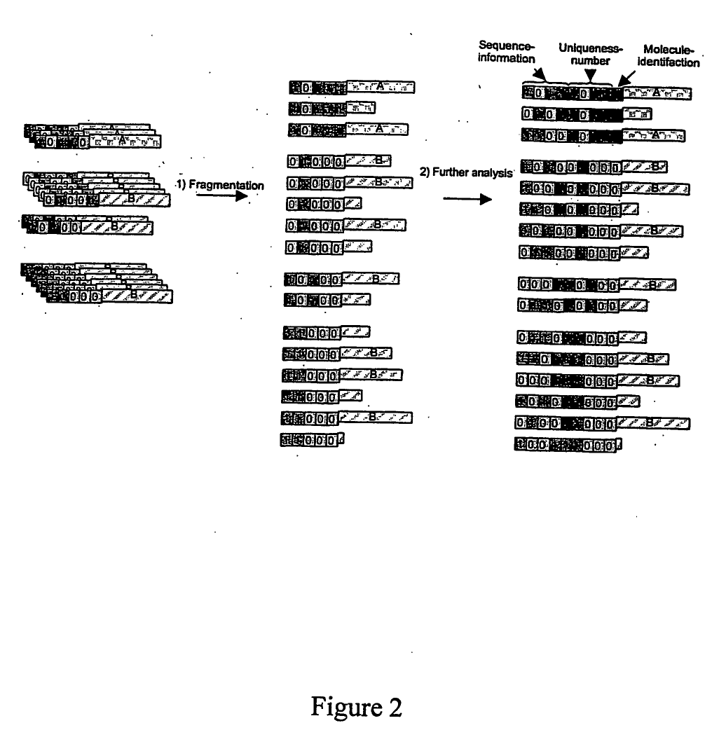

According to a further aspect of the invention, the sequence of a polynucleotide in a sample may be determined, for example in de novo sequencing. This aspect is illustrated by FIG. 2.

A molecular tag is attached to substantially all of the polynucleotides in the sample, as described previously. The sample polynucleotides are then fragmented, by methods well known in the art, for example as disclosed in WO-A-00/39333, the content of which is hereby incorporated by reference. At least the fragments which comprise a tag may then be sequenced, using methods of polynucleotide sequencing well known in the art. Since there will now be a collection of tagged polynucleotide fragments that, collectively, represent the entire sequence of the original sample molecules, and the origin of each fragment is known due to the tag, re-assembly of the sequence data is simplified.

In a preferred embodiment, the magnifying tag method of sequencing is used, as disclosed in WO-A-00/39333 the content of which is incorporated by reference. This describes a method for sequencing polynucleotides by converting the sequence of a target polynucleotide into a second polynucleotide having a defined sequence and positional information contained therein. The sequence information of the target is said to be “magnified” in the second polynucleotide, allowing greater ease of distinguishing between the individual bases on the target molecule. This is achieved using “magnifying tags” which are predetermined nucleic acid sequences. Each of the bases adenine, cytosine, guanine and thymine on the target molecule is represented by an individual magnifying tag, converting the original target sequence into a magnified sequence. Conventional techniques may then be used to determine the order of the magnifying tags, and thereby determining the specific sequence on the target polynucleotide. Each magnifying tag may comprises a label, e.g. a fluorescent label, which may then be identified and used to characterise the magnifying tag.

Another preferred method of sequencing is disclosed in WO-A-2004/094663, the content of which is hereby incorporated by reference. This is based on the “magnifying tags” method of sequencing, wherein the target polynucleotide sequence is converted into a second “magnified” polynucleotide. The second polynucleotide is then contacted with at least two of the nucleotides dATP, DTTP, dGTP and DCTP wherein at least one nucleotide comprises a specific detectable label, in order to allow rapid determination of the sequence of the target polynucleotide.

The tracking of the various stages of the analysis procedure(s) may be carried out using computer means. For example, after each reaction, the molecular tag can be identified and the characteristic(s) of the target molecule associated with the molecular tag stored in a computer. Subsequent reactions using the target molecule can be carried out and the further results determined and associated with the molecular tag. This information may also be stored, resulting in the collation of various reaction results for a specific target molecule.

1. A method of quantifying the absolute or relative number of molecules present in a sample after carrying out an analysis procedure on the sample, comprising the steps of:
(i) attaching a unique molecular tag to substantially all of the molecules in the sample;
(ii) carrying out the analysis procedure using the molecules of the sample; and
(iii) on the basis of the molecular tags determining the absolute or relative number of molecules present in the original sample which underwent the analysis procedure.

2. A method according to claim 1, further comprising, either before or after analysis, the step of incorporating into the molecular tag a sample identification portion.

3. A method according to claim 1, wherein step (iii) is carried out by identifying the tag in a read-out step.

4. A method according to claim 3, wherein the read-out step is carried out in a manner that ensures that each tag in the original sample is read at least once.

5. A method according to claim 1, wherein the molecules are polymer molecules.

6. A method according to claim 1, wherein the sample comprises different molecules.

7. A method according to claim 1, wherein the sample comprises multiple molecules of the same type.

8. A method according to claim 1, wherein the molecular tag is or comprises a polynucleotide molecule of defined sequence.

9. A method according to claim 8, wherein the polynucleotide is a DNA molecule of defined sequence.

10. A method according to claim 1, wherein the molecular tag is or comprises an antibody.

11. A method according to claim 1, wherein the molecular tag is or comprises an aptamer.

12. A method according to claim 1, wherein the molecular tags are polynucleotides and the analysis procedure involves an amplification reaction.

13. A method according to claim 1, wherein the polynucleotide tags are amplified in a polymerase reaction.

14. A method according to claim 13, wherein the molecules are polynucleotides and the analysis procedure involves an amplification of the polynucleotide molecules.

15. A method according to claim 14, wherein two or more polynucleotide molecular tags are bound to each target polynucleotide, and said tags subsequently ligated together and the resulting ligated polynucleotide amplified in a polynucleotide amplification reaction.

16. A method according to claim 1, wherein the analysis procedure involves nano-pore detection.

17. A method according to claim 1, wherein the molecular tag, or a part of the molecular tag, indicates the sample-origin of the tagged molecule.

18. A method according to claim 1, wherein the results of step (iii) are collated in a computer programme.

19. A method according to claim 1, wherein the molecules are proteins.

20. A method according to claim 18, wherein the molecules are antibodies.

21. A method for detecting the presence of a molecule in a sample, comprising contacting the sample with two or more molecule-binding moieties each having affinity for different parts of the target molecule, wherein the moieties comprise a polynucleotide molecular tag and wherein, on binding of at least two moieties to the target molecule, two or more molecular tags are ligated in a subsequent ligation step, and the ligated polynucleotide detected, characterised in that the ligated polynucleotide comprises a sequence that identifies the class of target molecule and the individual molecule.

22. A method according to claim 21, wherein the ligated polynucleotide further comprises a sample identification portion.

23. A method for detecting the presence of specific molecules present on the outer-surface of a cell or membrane, comprising:
(i) contacting the cell or membrane with a sample comprising different molecule-targeting moieties, each moiety comprising a polynucleotide molecular tag of defined sequence;
(ii) carrying out a ligation reaction to ligate adjacent polynucleotides; and
(iii) detecting the ligated polynucleotide(s) and determining the presence of the outer-surface or membrane molecules;
wherein the polynucleotide molecular tags comprise a nucleotide sequence that identifies the class of outer-surface molecule and optionally the individual molecule.

24. A method according to claim 23, wherein the polynucleotide molecular tag further comprises a sample identification portion.

25. A method according to claim 19 or claim 20, wherein the outer surface molecule is a protein, and the moiety is a protein-binding molecule.

26. A method according to any one of claims 8, 9 or 21 to 23, wherein the polynucleotide molecular tag comprises a sequence of nucleotides representing distinct units of binary code.

27. A method for determining the sequence of a polynucleotide in a sample, comprising the steps of:
i) attaching a unique molecular tag to polynucleotides in the sample;
ii) amplifying the polynucleotides;
iii) fragmenting the amplified polynucleotides; and
iv) sequencing at least those fragmented polynucleotides that comprise a molecular tag and identifying the molecular tag wherein, on the basis of the molecular tags, the sequence information for each individual polynucleotide is collated.

28. A method according to claim 27, wherein the molecular tag is as defined in any one of claims 9 to 12 or claim 17.

29. A method according to claim 22 or claim 23, wherein the sequencing step comprises converting the sequence information into magnifying tags, each tag representing one base in the polynucleotide.

30. A method according to any one of claims 22 to 24, wherein the results of step (iv) are collated in a computer programme.

31. A method for determining the sample origin of a biological molecule, comprising labelling the biological molecule with a molecular tag that is specific for the sample from which the molecule was taken or placed into, wherein, the sample origin is determined by identifying the molecular tag.

32. A method according to claim 31, wherein the molecular tag is as defined in any one of claims 9 to 12 or claim 17.

33. A kit comprising a discrete compartment comprising one or more molecular tags as defined in any one of claims 9 to 12 or claim 17.
